(19) United States
(12) Patent Application Publication
(10) Pub. No.: US 2013/0060254 A1
(43) Pub. Date: Mar. 7, 2013

(54) EYE MANIPULATOR

(71) Applicants: Tibor Juhasz, Corona del Mar, CA (US); Laszlo Nagy, Newport Beach, CA (US)

(72) Inventors: Tibor Juhasz, Corona del Mar, CA (US); Laszlo Nagy, Newport Beach, CA (US)

(21) Appl. No.: 13/223,805

(22) Filed: Sep. 1, 2011

(51) Int. Cl. A61F 9/00

(52) U.S. Cl. 606/107

(57) ABSTRACT

An eye-manipulator can include an applicator-arm and an eye-contact element, the applicator-arm providing mechanical control over the eye-contact element, and the eye-contact element, connected to the applicator-arm, can be configured to make contact with an eye. A method of manipulating an eye includes aligning the eye-contact element with an eye by adjusting the applicator-arm of the eye-manipulator; contacting the eye with the eye-contact element of the eye-manipulator; and manipulating the eye with the eye-manipulator to assist a docking of a patient interface of an ophthalmic system onto the eye.

### TECHNICAL FIELD

This patent document relates to an auxiliary device for ophthalmic procedures. In more detail, the auxiliary device can include an eye-manipulator to enable a surgeon to control and manipulate an eye during ophthalmic procedures, including cataract surgeries.

### BACKGROUND

The widespread introduction and acceptance of laser surgical systems in ophthalmic applications ushered in a new era of precision and control. One of the keys to achieving this high level of control is the immobilization of the eye relative to the laser surgical system. In many devices, the immobilization is carried out by affixing a patient interface to the objective of the laser and then docking the patient interface onto the eye. The docking is often achieved by engaging a vacuum suction system.

One of the factors the precision and utility of these systems depends on is the patient interface being docked to the eye in a central position. Such a central docking, or centering, aligns an optical axis of the objective of the laser system and an optical axis of the eye. Since the surgical laser beam is typically directed and controlled relative to the optical axis of the objective, aligning the optical axis of the eye with the optical axis of the objective by a central docking can enable controlling and directing the laser beam in the eye without needing any corrections for a misalignment. Further, the aberrations of the optic of the laser system are greater for target points farther from the optical axis: therefore a centered optic minimizes the aberrations as well as increases the precision.

Achieving a central docking is often a challenge, though, for multiple reasons. The patients sometimes move their eyes during docking, even against their own will. In other cases, the globe of the eye often rolls to the side during the docking process because of the pressure applied by the patient interface to the slippery surface of the globe.

Finally, the shape of most eyes is irregular to some degree. For example, often the pupil is not entirely circular, or the limbus and the pupil are not concentric. In these typical cases the center of the eye is not entirely well-defined. Therefore, centering the patient interface with the pupil may not center it relative to the limbus, even less with the lens, an internal eye structure and the target of cataract procedures.

### SUMMARY

The difficulties of docking the patient interface onto the eye can be reduced by manipulating the eye mechanically while the objective of the surgical laser is being aligned with and lowered onto the eye. Ophthalmic surgeons often manually rotate or move the eye into a desired position to center the docking of the patient interface.

However, direct manual manipulations of the eye still have only limited precision. Also, the eye can still move or rotate around a bit while being handled by the surgeon. In addition, for many ophthalmic surgical systems, space is very tight above the eye. Thus, even if the surgeon is capable of rotating the eye manually into a desired position with his or her fingers when the objective and the patient interface (PI) are at a distance, when the PI is being lowered onto the eye, there may not be sufficient space for the surgeon's fingers to keep holding the eye in the aligned position anymore.

The listed sources of imprecision of the direct manual alignment of the eye are particularly problematic in light of the increasing precision required by today's increasingly sophisticated ophthalmic laser systems. For example, some laser systems have a nominal precision of directing their laser beam by a precision of 5-10 microns or less. To realize this nominal precision, a correspondingly tight centering of the PI with the eye may be required.

In addition, new types of ophthalmic procedures, such as cataract procedures are attempted by laser systems, requiring further increases in precision. For example, during the capsulotomy phase of a cataract procedure, a circular cut is formed in the anterior portion of the lens-capsule of the eye. To avoid creating and leaving behind a visible circle in the patient's field of vision, the capsulotomy cut typically tracks the outer perimeter of the lens capsule by only about 0.5 mm, forming a rim. A decrease of the width of the rim by as little as 0.1 mm can make the rim too weak and thus susceptible to tearing, whereas even a small increase may create and leave a visible line in the patient's field of vision. Placing a cut with such a narrow tolerance may require centering the PI with the eye with a precision of smaller than 0.1 mm, i.e. smaller than 100 microns.

The above described high precision, required by the new generation of laser systems and the new types of ophthalmic procedures, however, may not be attainable in systems where the eye is aligned only manually.

The precision of the manual alignment of the eye can be improved by employing a gantry in the laser system to repeatedly move the objective laterally as it is lowered onto the eye. However, the repeated adjusting of the gantry can be a slow and challenging procedure. Further, the lateral adjustment of the objective with a gantry cannot compensate for a rotational or angular misalignment of the eye with the patient interface, which therefore is still compensated manually.

In some systems, this problem is addressed by employing a two piece patient interface. A contact portion of these patient interfaces can include an applanation or contact lens, a gripper and a vacuum suction ring. The gripper portion of the two piece PI allows the surgeon to manually align the contact lens with the eye, lower the contact lens until contact between the applanation lens and the eye is established, and then dock the applanation lens onto the eye by applying vacuum suction to the vacuum suction ring. A second portion of the patient interface can be affixed to the objective, and then the surgeon can align and lock the two portions together.

However, the optical precision of such systems can be limited because part of their optical train, the contact lens, is coupled to the rest of the optic only manually by the surgeon and not by a high-precision manufacturing process. Moreover, after the PI is docked to the eye, the gripper portion of the two piece PI remains attached to the eye during the laser procedures. Since the gripper portion of the two piece PI extrudes asymmetrically in a particular direction, it can disadvantageously exert a deforming force and torque on the eyeball, as well as block the surgeon's access to the eye.

Embodiments of an eye-manipulator offer improvements and solutions to the above described problems. In particular, an embodiment of an eye-manipulator can include an eye-contact element, configured to contact an eye to provide control over an alignment of the eye, and an applicator-arm, coupled to the eye-contact element to provide mechanical control over the eye-contact element, wherein the eye-contact element is not configured to accommodate an optical lens.

In other embodiments, a method of manipulating an eye can include aligning an eye-contact element of an eye-manipulator with an eye by manipulating an applicator-arm of the eye-manipulator, contacting the eye with the eye-contact element, and manipulating the eye with the eye-manipulator to assist a docking of a patient interface of an ophthalmic system onto the eye, wherein the eye-contact element is not configured to accommodate an optical lens.

In some embodiments, an eye-manipulator can include an eye-contact element, and an applicator-arm, connected to the eye-contact element to provide mechanical control over the eye-contact element, wherein the eye-contact element is configured to contact an eye to provide control of an alignment of the eye, is separate from a patient interface of an ophthalmic surgical system, and is removable from the eye after the patient interface is docked to the eye.

### BRIEF DESCRIPTION OF THE DRAWINGS

FIG. 1 illustrates an ophthalmic laser system.

FIGS. 2A-B illustrate top views of embodiments of an eye-manipulator.

FIG. 3 illustrates a side view of an eye-manipulator.

FIGS. 4A-B illustrate embodiments of the contact pads.

FIGS. 5A-B illustrate the stages of manipulating and aligning the docking eye and docking the patient interface onto the aligned docking eye.

FIG. 6 illustrates a method of manipulating an eye.

### DETAILED DESCRIPTION

Implementations and embodiments in this patent document provide an eye-manipulator that can improve the precision of the docking of a patient interface onto an eye.

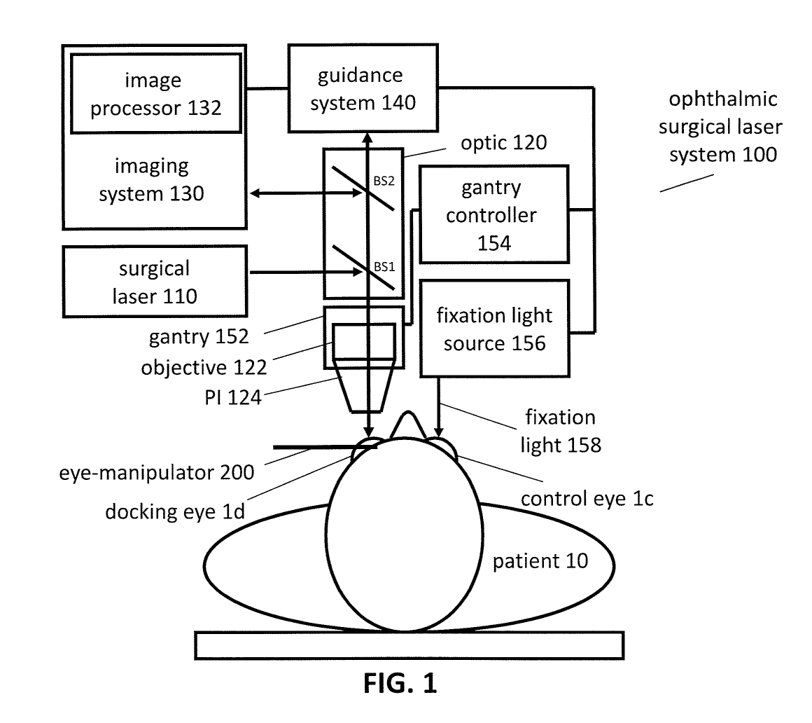

FIG. 1 illustrates an ophthalmic surgical laser system 100. The laser system 100 can include a surgical laser 110 that can generate and couple a surgical laser beam into an optic 120 at a beam splitter BS1. The surgical laser 110 can be capable of generating a pulsed laser beam with a femtosecond or picosecond pulse length. The optic 120 can redirect and deliver the pulsed laser beam into a docking eye 1d of a patient 10 through an objective 122 and a patient interface (PI) 124.

The laser system 100 can also include an imaging system 130. The imaging system 130 can provide one or more images for the ophthalmic surgeon to increase the precision of the ophthalmic procedure. The image can include a stereoscopic microscope image, a video-image, a Scheimpflug image, or an Optical Coherence Tomographic (OCT) image. The image can be analyzed by an image processor 132.

The generated image can be displayed on a guidance system 140. One of the functions of the guidance system 140 can be to guide the surgeon to align a center of the eye and a center or axis of the optic 120 for optimizing the docking. In some embodiments, the guidance system 140 can include a video-monitor to display the video image. In others, the guidance system can include an OCT display to display the OCT image created by the imaging system 130. In yet others, the guidance system 140 can display both a video image and an OCT image.

In addition, the guidance system 140 can include a guidance display to guide the surgeon based on the result of the processing of the image by the image processor 132. For example, the guidance display of the guidance system 140 can include a target pattern or a crosshair pattern overlaid on the video image of the eye to indicate a position of an optical center or axis of the optic 120, thus allowing the surgeon to determine the position of the eye relative to the axis of the optic 120. In other systems, the guidance system 140 can display one or more arrows to suggest the surgeon a corrective action to align the optic 120 and the eye. In yet other systems, the guidance system can display aligning icons determined from an analysis of the OCT image by the image processor 132.

The correction of the alignment can be initiated either by the surgeon or by a processor of the surgical laser system 100, in response to the guidance information generated by the guidance system 140. For example, some embodiments of the laser system 100 can include a gantry 152 and a gantry controller 154 to move the objective 122 laterally and align it with a center of the eye as part of the docking procedure. The movement of the gantry 152 can compensate a lateral or transverse misalignment of the eye and the optic 120, but not a rotational misalignment.

A rotational or angular misalignment of the eye and the optical axis of the optic 120 can be compensated by a fixation light source 156 that projects a fixation light 158 into a control eye 1c. The patient 10 can be instructed to follow the movement of the fixation light 158. As the surgeon adjusts the fixation light 158, he or she can follow the movement of the video image of the eye relative to the optical axis of the optic 120 on the guidance display and continue to adjust the fixation light 158 until the docking eye 1d is aligned with the optical axis of the optic 120 to the desired degree.

However, both the lateral adjustment of the gantry 152 and the rotational alignments caused by the adjusting of the fixation light 158 can be slow and ineffective, leading to an alignment of the docking eye 1d and the objective 122 with limited precision only.

As described above, if complementary direct manual manipulations of the docking eye 1d are attempted to enhance the precision of the alignment with a gantry and a fixation light, those manipulations can introduce both advantages but also new types of limitations, thus not necessarily improving the overall precision.

To address these problems, embodiments of the present invention include auxiliary eye-manipulator devices that allow the surgeon to manipulate the docking eye 1d with a precision higher than that of a direct manual manipulation. These eye-manipulators also allow the surgeon to keep the docking eye 1d in the aligned position as the objective 122 and the PI 124 are lowered onto it, eliminating the requirement for extra space for the fingers of the surgeon in the crowded surgical space. These eye-manipulators can be utilized well to complement laser systems with one-piece PIs 124, where a gripper-type architecture is not being used, for example.

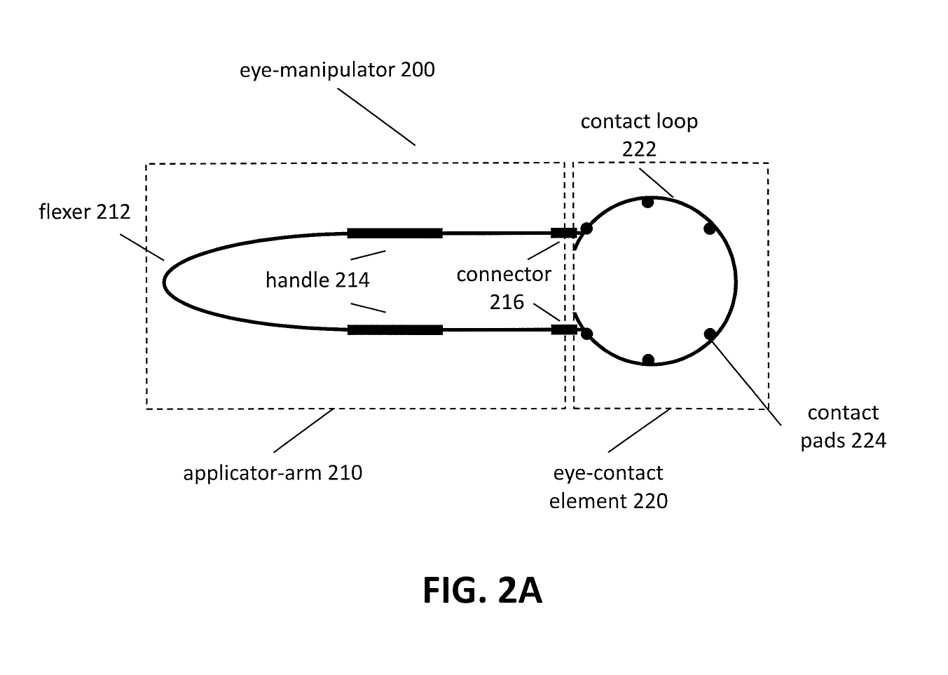

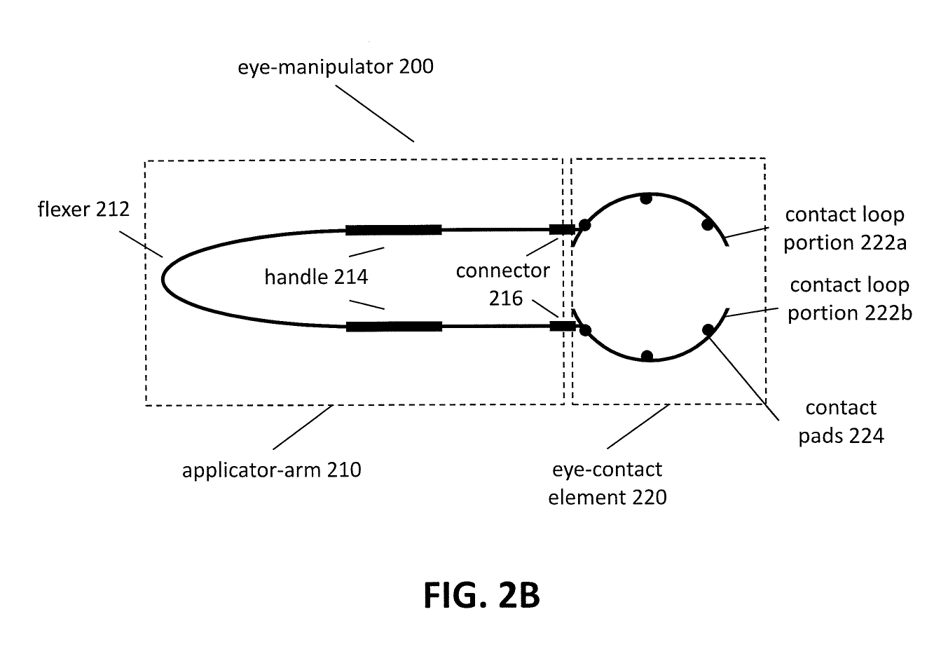

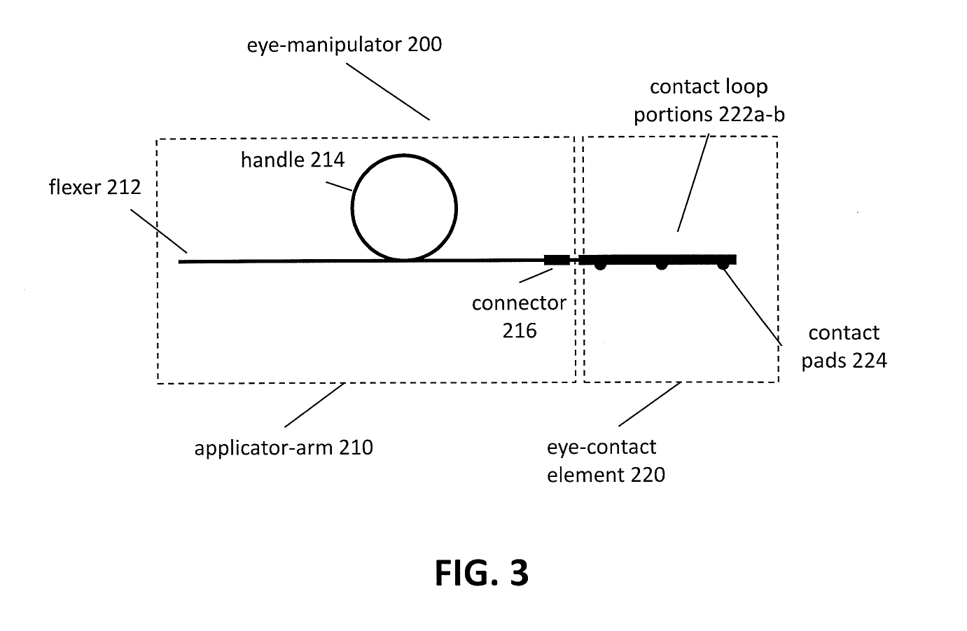

FIGS. 2-3 illustrate embodiments of an eye-manipulator 200 that can include an applicator-arm 210 and an eye-contact element 220. The applicator-arm 210 can provide mechanical control over the eye-contact element 220. The eye-contact element 220 can be coupled to the applicator-arm 210 and can be configured to make contact with the docking eye 1d to provide control over an alignment of the docking eye 1d.

Aspects of the eye-manipulator 200 include that the eye-contact element 220 may not be configured to accommodate an optical element such as an optical lens. This aspect has been discussed earlier: the gripper portion of the two piece PIs can accommodate a contact lens either by directly having it in the gripper, or by having an opening into which the contact lens can be inserted. Since the other portion of the two piece PI, attached to the objective 122, is coupled to the gripper portion by the surgeon manually and under the time pressure of the surgery, the optical precision by which the beam is directed by such two-piece PIs can be limited.

In contrast, embodiments of the eye-manipulator 200 may not include or accommodate an optical lens: all optical elements of the optical train of the ophthalmic surgical laser system 100 can be assembled during the high precision manufacturing process or during the preparation for the surgery, thus without time pressure, and therefore can achieve the high precision required by today's modern laser systems as well as by the advanced surgical procedures.

Further, embodiments of the eye-manipulator 200 can be separate from the PI 124 and not attach to any part of the PI 124. Therefore, embodiments of the eye-manipulator 200 can be removed from the docking eye 1d after the PI 124 has been docked to the eye 1d. This characteristic can be useful as an eye-contact element 220 that remains attached to the eye after docking can exert a deforming torque or force on the eye as well as obstruct the surgeon's actions by keeping the surgical space crowded.

In addition, the thickness of the eye-contact element 220 can be considerably thinner than the thickness of a human finger or of some grippers. Therefore, as the PI 124 is lowered onto the docking eye 1d, it can be lowered considerably past the point where during the direct manual manipulation the surgeon would have to remove or slide out his fingers from the eye, thus potentially undermining the precision of the docking procedure.

In contrast, because of its thinness, the eye-contact element 220 may not need to be removed until the docking is actually completed. This feature is a further aspect of the eye-manipulator 200 increasing the precision of the alignment in comparison to the direct manual manipulation.

The thinness of the eye-contact element 220 also provides a much clearer and precise visual control for the surgeon. In systems that require direct manual manipulations of the eye, the fingers of the surgeon can block a substantial portion of the video image of the eye, and the point of contact between the fingers and the eye may not even be visible on the video image. In contrast, the thin eye-contact element 220 may block very little of the video image and its contact point with the eye is well defined. In particular embodiments, the thickness of the eye-contact element 220 can be less than 5 mm, in some other embodiments less than 2 mm.

In some embodiments, the applicator-arm 210 can include a flexer 212 to provide a spring action for portions of the eye manipulator 200. For example, in FIG. 2A, the eye-contact element 220 can have a contact loop 222 that is flexible and partially open. FIG. 2B illustrates another embodiment where the eye-contact element 220 can include two partial half-rings or loop portions 222a-b. In either embodiment, the contact loop 222 or contact loop portions 222a-b can define a radius larger than a contact or application radius. For such embodiments, the surgeon has the option of pressing together the two arms of the flexer 212 to flexibly reduce the radius of the contact loop 222 or loop portions 222a-b to the application radius when engaging the eye-contact element 220 with the docking eye 1d. Using such a flexer 212 can enhance the force pressing the eye-contact element 222 to the eye 1d and thus increase the control provided for the surgeon by the eye-manipulator 200. Some embodiments may have sufficient contact force and may not need such a flexer 212 to be sufficiently useful.

FIG. 3 illustrates a side view of some embodiments where the applicator-arm 210 can include a handle 214 to allow an operator the above described mechanical control over the eye-contact element 220. Indeed, the applicator-arm 210 with the handle 214 can form a useful and practical combination to provide comfortable control for the surgeon over the precise placement and shape of the eye-contact element 220.

FIGS. 2A-B illustrate in a top view and FIG. 3 in a side view that in some embodiments of the eye-manipulator 200 the applicator-arm 210 can include a wire, where the flexer 212 includes a bent portion of the wire and the handle 214 includes two looped portions of the same wire. Creating the eye-manipulator 200 by deforming and shaping a single wire to form a flexible bend 212 and two looped handles 214 allows an efficient and cheap manufacturing of the eye-manipulator 200. Other embodiments of the eye-manipulator 200 can be formed by molding a plastic, or by combining plastic and metal components, or by using elastic materials.

In some embodiments, the eye-contact element 220 can include the flexible contact loop 222 or two contact loop portions 222a-b. A function of this contact loop 222 or loop portions 222a-b can be to form the actual physical contact with the docking eye 1d, thus enabling the manipulations of the eye without the surgeon placing fingers directly on the eye or eye globe.

Embodiments with the two contact loop portions 222a-b allow the adjusting of the radius of the eye-contact element 220 in a considerable range. This can be useful, since there are considerable variations of the eye radii from patient to patient depending on age, medical condition and geography, among others.

Embodiments with the movable contact loop portions 222a-b also allow the release of the grip of the eye-manipulator 200 after the docking of the PI 124 is completed and the subsequent removal of the eye-manipulator 200 as the eye-manipulator 200 is not part of the PI 124, but rather, it is separate from the PI 124. The eye-manipulator 200 can be removed from the surgical area by letting the arms of the applicator-arm 210 to spread wider, thus distancing the contact loop portions 222a-b or increasing the radius of the flexible loop 222 of the eye-contact element 220. In embodiments with the contact loop portions 222a-b the distance of the portions 222a-b can be increased to a degree that the eye-manipulator 200 can be removed from the surgical area in its entirety. In embodiments with the flexible contact loop 222 the radius of the loop 222 can be broadened so that the eye-contact element 200 is removed from the area partially in the sense that it does not interfere with the subsequent surgical procedures. Removing the eye-manipulator 200 from the eye fully or partially can clear and thus simplify the surgical space for the surgeon as well as it can eliminate deforming torques and forces on the docking eye 1d. In contrast, some existing gripper portions of two piece PIs must remain attached to the eye as they hold the contact lens that is part of the optical train. Since these gripper portions of the two piece PIs remain in the surgical area, they continue to clutter the surgical area and exert deforming torques and forces on the eye.

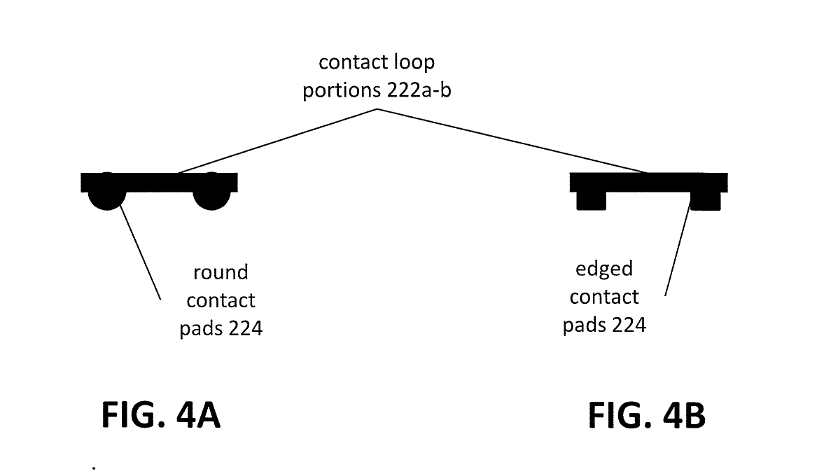

FIGS. 4A-B illustrate that in some embodiments, the eye-contact element 220 can include contact pads 224, attached to the contact loop 222 or contact loop portions 222a-b. These contact pads 224 may substantially reduce the actual contact area between the eye-contact element 220 and the docking eye 1d, and therefore considerably increase the pressure on the eye tissue. Increased pressure can make the contact pads 224 depress the eye tissue deeper compared to a pad-free contact loop 222. Such a deeper depression can generate a stronger frictional force and a stronger grip, thus providing better mechanical control of the eye by the eye-manipulator 200.

FIG. 4A illustrates embodiments with round contact pads 224. FIG. 4B illustrates contact pads 224 with edges, providing high pressure contact areas, possibly exceeding the pressure generated by the round contact pads 224. Finally, in some embodiments the contact loop 222 or contact loop portions 222a-b can have a roughened surface to increase the frictional force with the eye.

Many embodiments of the eye-contact element 220 can include a bio-compatible material to facilitate the direct contact with the eye-tissue.

Some embodiments of the eye-manipulator 200 can include a connector 216 to connect the applicator-arm 210 and the eye-contact element 220 removably. For example, since only the eye-contact element 220 makes direct contact with the eye itself, the applicator-arm 210 can be reusable. In such embodiments, the applicator-arm 210 may include or end in a pair of connectors 216, into which new and sterile eye-contact elements 220 can be inserted before new procedures.

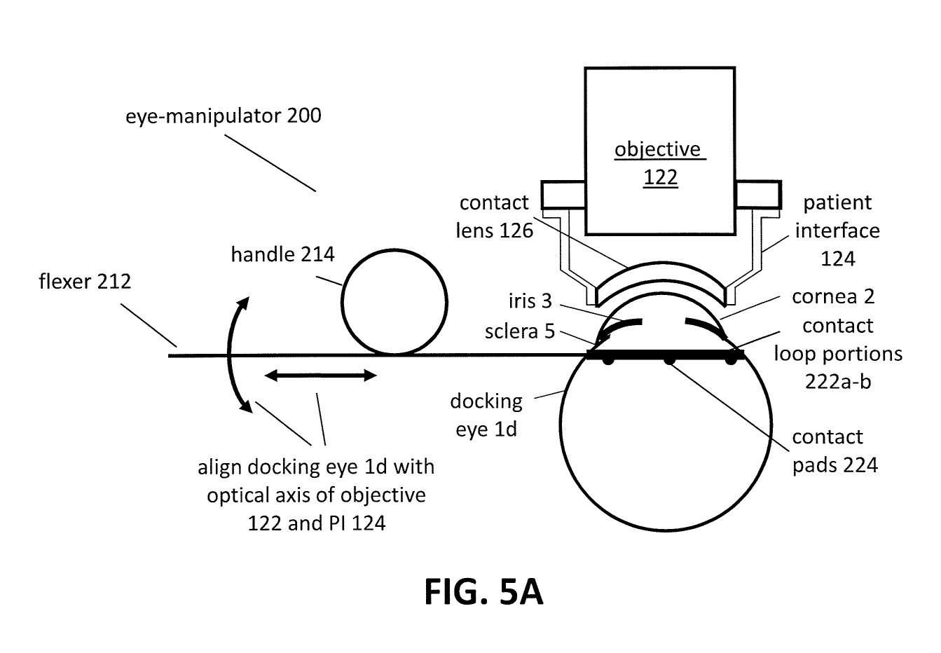

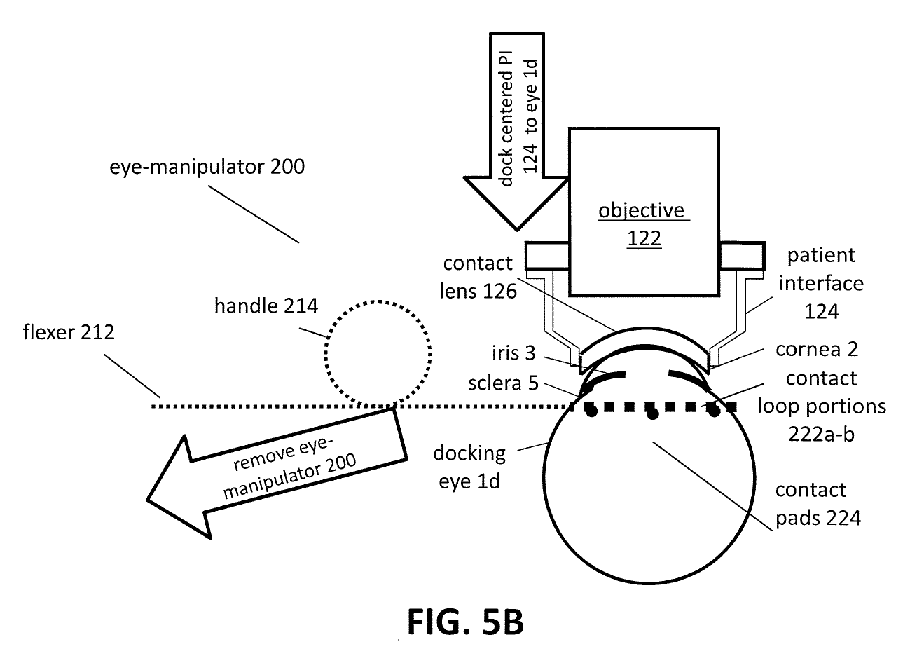

FIGS. 5A-B illustrate an embodiment of the eye-manipulator 200 during a docking procedure. In FIG. 5A the eye-manipulator 200 has engaged the docking eye 1d and allows the surgeon to control the docking eye 1d to align its optical axis with the optical axis of the objective 122 and the PI 124 by manipulating the eye-manipulator 200.

FIG. 5B illustrates that once a high degree of alignment of the docking eye 1d, the objective 122 and the PI 124 is reached, the centered or aligned patient interface 124 with a contact lens 126 can be lowered and docked onto the docking eye 1d in a centered position. Since the eye-manipulator 200 is not part of the PI 124 and does not contain the contact lens 126, after the docking the eye-manipulator 200 can be removed from the surgical space, partially or in its entirety.

As discussed before, since the one piece PI's contact or applanation lens 126 is not part of the eye-manipulator 200, it can be accommodated into the one piece PI 124 and subsequently calibrated with a high precision during the manufacturing process. Further, the one piece PI 124 can have a precision engaging system for the attachment to the objective 122 that is much more precise than that of a gripper portion of a two piece PI. Finally, since the contact lens 126 is attached to the PI 124 during manufacturing and since the PI 124 is attached to the objective 122 prior to the surgery and thus without the concomitant time pressure, the overall alignment of the contact lens 126 with the optical axis of the objective 122 can be considerably more precise than with two piece PIs with a gripper, as demanded by the new generation of surgical laser systems and high precision surgical procedures.

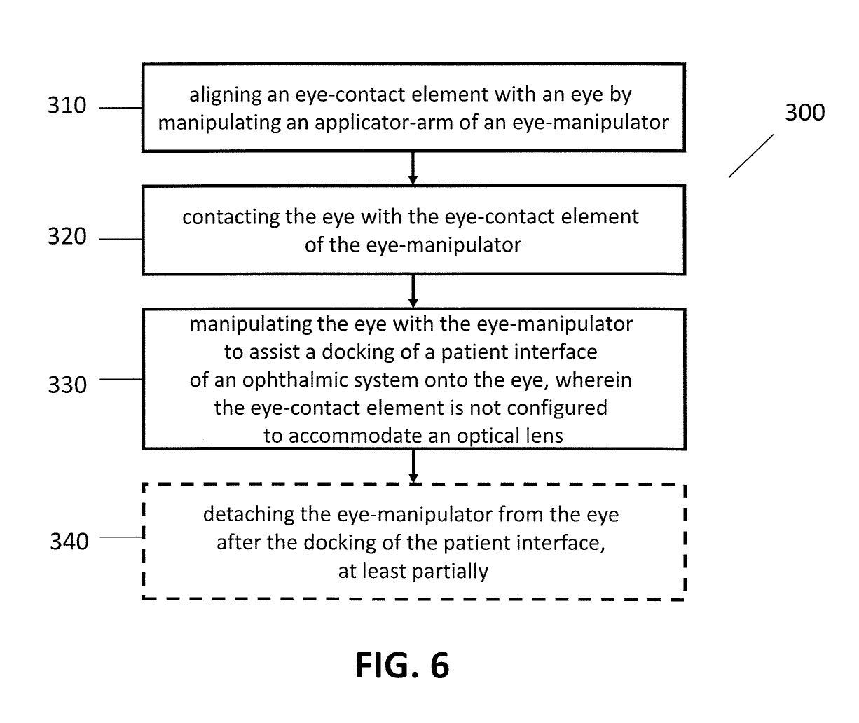

FIG. 6 illustrates that in some embodiments, a method 300 of manipulating an eye can include an aligning 310 of an eye-contact element of an eye-manipulator with an eye by adjusting an applicator-arm of the eye-manipulator; a contacting 320 of the eye with the eye-contact element of the eye-manipulator; and a manipulating 330 of the eye with the eye-manipulator to assist a docking of a patient interface of an ophthalmic system onto the eye.

Here the eye-manipulator can be the eye-manipulator 200, the applicator-arm can be the applicator-arm 210, the eye-contact element can be the eye-contact element 220, and the patient interface can be the patient interface 124 of the ophthalmic surgical laser system 100.

In some embodiments, the eye-contact element 200 may not be configured to accommodate an optical lens, such as the contact lens 126 of the patient interface 124.

In some embodiments, the method 300 can also include a detaching 340 of the eye-manipulator from the eye after the docking of the patient interface, at least partially.

While this specification contains many specifics, these should not be construed as limitations on the scope of the invention or of what can be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

What is claimed is:

1. An eye-manipulator, comprising:
an eye-contact element, configured to contact an eye to provide control over an alignment of the eye; and
an applicator-arm, coupled to the eye-contact element to provide mechanical control over the eye-contact element, wherein
the eye-contact element is not configured to accommodate an optical lens.

2. The eye-manipulator of claim 1, the applicator-arm comprising:
a flexer, configured to provide a spring action for portions of the eye-manipulator, wherein
the eye-manipulator has portions.

3. The eye-manipulator of claim 1, the applicator-arm comprising:
a handle, configured to provide an operator the mechanical control over the eye-contact element.

4. The eye-manipulator of claim 1, the applicator-arm comprising:
a wire, wherein
the flexer includes a bent portion of the wire; and
the handle includes two looped portions of the wire.

5. The eye-manipulator of claim 1, the eye-contact element comprising:
a contact loop.

6. The eye-manipulator of claim 1, the eye-contact element comprising:
two contact loop portions.

7. The eye-manipulator of claim 1, the eye-contact element comprising:
contact pads, configured to provide increased-pressure contact areas with the eye.

8. The eye-manipulator of claim 7, wherein:
the contact pads have edges, configured to provide high-pressure contact areas with the eye.

9. The eye-manipulator of claim 1, the eye-contact element comprising:
a roughened surface to provide a high frictional force contact area with the eye.

10. The eye-manipulator of claim 1, the eye-contact element comprising:
a bio-compatible material.

11. The eye-manipulator of claim 1, comprising:
a connector, configured to connect the applicator-arm and the eye-contact element removably.

12. A method of manipulating an eye, the method comprising:
aligning an eye-contact element of an eye-manipulator with an eye by manipulating an applicator-arm of the eye-manipulator;
contacting the eye with the eye-contact element; and
manipulating the eye with the eye-manipulator to assist a docking of a patient interface of an ophthalmic system onto the eye, wherein
the eye-contact element is not configured to accommodate an optical lens.

13. The method of claim 12, the method comprising:
detaching the eye-manipulator from the eye after the docking of the patient interface, at least partially.

14. An eye-manipulator, comprising:
an eye-contact element; and
an applicator-arm, coupled to the eye-contact element to provide mechanical control over the eye-contact element, wherein
the eye-contact element is
configured to contact an eye to provide control of an alignment of the eye,
separate from a patient interface of an ophthalmic surgical system, and
removable from the eye after the patient interface is docked to the eye.

15. The eye-manipulator of claim 14, the eye-contact element comprising:
a loop or loop-portions.

16. The eye-manipulator of claim 14, the eye-contact element comprising:
contact pads.

17. The eye-manipulator of claim 14, wherein:
the eye-contact element is not configured to accommodate an optical element.

18. The eye-manipulator of claim 14, the applicator-arm comprising:
a spring-action element; and
a handle, configured to provide control over the eye-contact element.
